(19) United States
(12) Patent Application Publication
(10) Pub. No.: US 2014/0127300 A1
(43) Pub. Date: May 8, 2014

(54) ABUSE RESISTANT DRUG FORMS

(71) Applicant: Neos Therapeutics, LP, Grand Prarie, TX (US)

(72) Inventors: Mark Tengler, Colleyville, TX (US); Russell McMahen, Flower Mound, TX (US)

(73) Assignee: Neos Therapeutics, LP, Grand Prarie, TX (US)

(21) Appl. No.: 14/128,750

(22) PCT Filed: Jul. 2, 2012
(86) PCT No.: PCT/US12/45255

§ 371 Date: Dec. 23, 2013

Related U.S. Application Data
(60) Provisional application No. 61/503,464, filed on Jun. 30, 2011.

(51) Int. Cl. A61K 31/485

(52) U.S. Cl. CPC A61K 31/485
USPC 424/487; 514/282

(57) ABSTRACT

The invention is directed to oral drug dosage forms designed to reduce the abuse potential of an oral dosage form of an opioid analgesic. The oral drug dosage form comprises a first population of drug-resin complex particles comprising an analgesically effective amount of an opioid drug, said first population of particles coated with a water-permeable diffusion barrier coating. The oral drug dosage form further comprises a second population of ion exchange-resin complex particles comprising an aversive agent, said second population of particles coated with a polymer coating sufficient to substantially prevent release of the aversive agent under normal use conditions. The aversive agent is present in an amount effective to partially or substantially deny the drug abuser the euphoric effect and/or cause an aversive effect in the user.

### BACKGROUND OF THE INVENTION

1. Field of the Invention

The invention is directed to drug forms designed to reduce the abuse potential of oral dosage forms of opioid drugs. The drug form comprises an analgesically effective amount of a controlled release opioid drug and an aversive agent which is not released, or not substantially released, in the body under normal use conditions. Physical alteration of the oral dosage form results in release of an amount of the aversive agent effective to partially or substantially deny the drug abuser the euphoric effect of the opiod and/or cause an aversive effect in the user. Drug forms comprising sustained release of the opioid drug, with or without an immediate release opioid drug component, are contemplated. The aversive agents may take the form of one or more opioid antagonists.

2. Description of Related Art

Opioids are a group of drugs that exhibit opium- or morphine-like qualities when administered to a patient. Opioids fall within a number of broad classes, including natural (e.g., morphine, codeine), semi-synthetic (e.g., hydromorphone, hydrocodone), and fully synthetic (e.g., fentanyl, methadone). Opioids bind to certain receptors in the central and peripheral nervous system, including the three central classes of opioid receptors known as mu (μ), kappa (κ), and delta (δ), depending on the opioid.

In the United States, opioids are generally prescribed for the following clinical indications: moderate to severe pain (including as an anesthetic during surgery); cough; and diarrhea. In the United States, buprenorphine maintenance therapy (BMT) and methadone maintenance therapy (MMT) is utilized for the management of opioid-dependent individuals.

Unfortunately, the latter highlights a problem in the United States with opioid abuse. Opioid drug abusers will attempt to obtain their opioid drug of choice for self-administration in order to achieve a “high.” For example, opioid drug abusers typically may take a controlled-release product containing an opioid analgesic and crush, shear, grind, chew, dissolve and/or heat, extract or otherwise damage the controlled-release product so that the opioid becomes available for immediate administration via injection, inhalation, and/or oral consumption. In the United States, efforts to control opioid drug abuse have included placing restrictions on the availability and use of opioids, such as for example in the treatment of pain in compulsive drug users.

Opioid drug abuse, and the efforts to address this problem, place a substantial burden on society. For example, society is forced to incur costs associated with preventive programs (i.e., educational programs), crime (both enforcement of existing laws as well as the unintended consequences of drug inhibition), absenteeism, sickness, and treatment. Thus, it is desirable for society to have medications available that address and/or deter opioid abuse, while reducing the burden opioid abuse places on society and the community of medical providers. For example, a sustained release dosage form of an opioid analgesic that further contains an abuse deterrent, such as an opioid antagonist and/or aversive agent, allows for the continued use of opioid analgesics for medically-approved indications while simultaneously reducing the incidence of drug abuse and/or the desire among abusers to participate in this behavior. Moreover, a sustained release form reduces the burden on the community of medical providers by reducing the frequency with which recipients must obtain prescriptions, for example. Where the medical indication involves the management of opioid-dependent individuals, a sustained release form may reduce the frequency with which the abuser seeks assistance from the medical community.

Sustained- or prolonged-release dosage forms provide a controlled supply of drug to an organism over an extended period of time. Oral controlled-release drug preparations may provide the convenience of daytime dosing where the dosage form can be administered to an animal first thing in the morning and provide therapeutic levels of the drug throughout the day. Further, an oral controlled-release drug preparation may deliver drugs in a manner that will maintain therapeutically effective plasma levels in a mammal over a period of time that is significantly longer than that which is given by a typical drug dosage form. This eliminates the need to interrupt sleep to take medication and can prevent missed doses, thus improving patient compliance. Benefits obtained from such a controlled release of a specific drug include the control of cough, sleep, enuresis, pain and migraine headaches. Additionally, controlled release of antimicrobials can be used to treat or prevent infection.

Liquid oral dosage forms are known in the art. Liquid forms have the distinct advantages of dosage flexibility and ease of swallowing. Moreover, there is a recognized need for sustained release forms to be available in a convenient, easy-to-take liquid dosage form. However, the form of liquid oral suspensions having sustained-released capabilities has only resulted in limited success. In part, this is due to the challenges presented in maintaining the stability of sustained-release particles when present in liquid dispersal systems, and the difficulty in achieving sustained release of the drug from the dispersed phase.

### Ion-Exchange Drug Resins

Ion-exchange technology has been an approach utilized for achieving sustained release for solid dosage forms, and various attempts have been made to further utilize the technology in liquid suspension forms as well. For example, U.S. Pat. No. 2,990,332 discloses a method of controlling the release rate of drug by adsorbing the salt form of a drug onto a carrier resin such as an ion-exchange resin. However, while complexing drugs on ion-exchange resins has been effective for taste-masking, such uncoated complexes provide only a relatively short delay of drug release and a poor control of drug release, because control of release rate is limited to variation in particle size and cross-linkage of the sulfonic acid-type resin used to prepare the adsorption compounds.

Another approach to prepare liquid suspensions having sustained-released capabilities is by coating drug resins with a water-permeable diffusion barrier. For example, U.S. Pat. No. 4,221,778 discloses a method for preparing products having controlled release properties involving a three-step process: (i) preparation of a drug-resin complex; (ii) treating this complex with a suitable impregnating agent; and (iii) coating the particles of treated complex with a water-permeable diffusion barrier. The impregnation agents are believed to act as humectants to stabilize the size of the swellable particle or minimize rupturing of the water-permeable diffusion barrier, and the barrier coating is believed to delay the release rate of the drug. U.S. Pat. Nos. 4,996,047 and 5,980,882 also provide drug-ion-exchange resin complex particles coated with a water-permeable diffusion barrier layer.

U.S. Pat. No. 4,762,709 discloses a form wherein a coated first drug/ion-exchange resin particle is suspended in a liquid carrier with an uncoated second drug/ion-exchange resin component bearing the same charge as the first drug in the coated first drug/ion-exchange resin particle. According to the reference, the release rate of the first drug from the coated first drug/ion-exchange resin particle is increased when the second drug is present in the second uncoated drug/ion-exchange resin complex compared to when the second drug is included with the first drug in the coated first drug/ion-exchange resin.

A product based on this ion-exchange technology is Tussionex® Pennkinetic° Extended-Release Suspension. Tussionex® drug suspension contains hydrocodone polistirex equivalent to 10 mg hydrocodone bitartrate and chlorpheniramine polistirex equivalent to 8 mg chlorpheniramine maleate. Tussionex® drug suspension was approved by the FDA in 1987.

Nevertheless, there remains a need for effective sustained-release opioid dosage forms designed to prevent or deter drug abuse while allowing for the continued use of opioid drugs for medically-approved indications. Preferably, such opioid dosage forms simultaneously reduce the incidence of drug abuse and/or the desire among abusers to participate in this behavior, while reducing the burden on the community of medical providers. The invention described herein is directed to meeting this need.

### BRIEF SUMMARY OF THE INVENTION

This invention is broadly directed to drug forms designed to reduce and/or deter the abuse potential of an oral dosage form of one or more opioid drugs, by including in the drug forms one or more aversive agents that are not released, or not substantially released, under normal use conditions. In one embodiment, the oral drug dosage form comprises a first population of sustained-release drug-containing resin particles comprising an analgesically-effective amount of an opioid drug bound to a water-insoluble, pharmacologically inert matrix, this first population of drug-resin complex particles further comprising a water-permeable diffusion barrier coating. The drug form further comprises a second population of aversive agent-containing resin particles comprising an aversive agent in an amount sufficient to deter abuse if tampered with, the second population of particles coated with a polymer coating which substantially prevents release of the aversive agent under normal use conditions. The aversive agent is present in an amount that, when released in response to tampering, is effective to substantially deny the drug abuser the euphoric effect of the opiod and/or cause an aversive effect in the user. In one embodiment of the invention, the aversive agent is an opioid antagonist.

In one embodiment of the invention, an aversive agent is sequestered in a form in which the aversive agent is not released under normal use conditions. Under normal use conditions of the body following oral administration, the aversive agent is not released, or not substantially released, into the subject from the oral dosage form.

In a non-limiting embodiment of the invention, the second population of aversive agent-containing resin particles comprising one or more aversive agents maintains the one or more aversive agents in a non-releasable, or substantially non-releaseable, form through the use of an ion exchange resin, which impedes the release of the one or more aversive agents through ionic binding. It is believed that the use of an ion-exchange resin confers a benefit over the use of a mere polymer coating, because the ion-exchange resin tightly binds the one or more aversive agents and slows their release. When combined with an appropriate polymer coating, the one or more aversive agents may be formulated to prevent release or prevent substantial release from the second population of aversive agent-containing resin particles. Only when exposed to certain ionic conditions will the one or more aversive agents begin to release from the ion exchange resin.

In another non-limiting embodiment of the invention, the second population of aversive agent-containing resin particles comprising one or more aversive agents maintains these agents in a non-releasable or substantially non-releaseable form through the inclusion of one or more chelating agents, which serve to prevent or delay changes in ionic conditions.

In another non-limiting embodiment of the invention, the second population of aversive agent-containing resin particles comprising one or more aversive agents maintains these agents in a non-releasable or substantially non-releaseable form through the use of a polymeric coating that prevents or delays changes in ionic conditions that could affect the release of bound aversive agents from the resin. The combination of two or even all three of these foregoing embodiments is contemplated.

Following physical alteration of the oral dosage form (i.e., tampering), an amount of the one or more aversive agents effective to substantially deny the drug abuser the euphoric effect and/or cause an aversive effect in the user is released.

In one embodiment of the invention, the first population of drug-resin complex particles comprises a water-permeable diffusion-barrier coating permitting sustained release of the opioid drug. In another embodiment of the invention, the first population of drug-resin complex particles comprises one or more coatings permitting both immediate release of a portion of the opioid drug and sustained release of a portion of the opioid drug.

### DETAILED DESCRIPTION OF PREFERRED EMBODIMENTS

### Definitions

It is to be understood that this invention is not limited to the particular methodology, protocols, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a particle” includes a plurality of such particles, and so forth. All technical and scientific terms used herein have the same meaning as commonly understood to one of ordinary skill in the art to which this invention belongs unless clearly indicated otherwise.

In the context of the present invention, by “finally formulated” means a suspension that includes all of the components typically formulated for commercial distribution such as stabilizers, thickeners, dyes, flavorants, etc.

As used herein, “substantially deny the drug abuser the euphoric effect” means that the aversive agent sufficiently blocks the analgesic and/or euphoric effect of the opioid drug to sufficiently reduce the potential for abuse of the opioid drug in the dosage form. Without being limited by theory, it is believed that certain opioid antagonists identified herein act as competitive inhibitors of opioid drugs, by binding to and blocking opioid receptors (such as for example the mu receptor). Such can be measured, for example, using surrogate measures such as a Visual Analogue Scale (“VAS”) for drug effect. See U.S. Pat. No. 6,696,088, incorporated herein by reference.

As used herein, “no significant release of the aversive agent” is intended to mean the release of an amount of aversive agent that does not affect the efficacy or tolerability of the drug.

As used herein, “normal use conditions” is intended to mean the administration of an intact pharmaceutical form to an individual without prior tampering or modification to the intact pharmaceutical form.

As used herein, two or more populations of particles that are “substantially similar in appearance” means that the two or more populations of particles are similar enough in size, color, appearance, texture, and other visible physical characteristics that it is difficult for an individual unaided eye to visibly differentiate between the two populations of particles.

In the context of the present invention, an “analgesically effective amount” is identified by one of skill in the art as being an amount resulting in the satisfactory reduction in, or elimination of, pain along with a tolerable level of side effects, as determined by the human patient, e.g., as measured by surrogate measures such as a Visual Analogue Scale (“VAS”) for drug effect.

A “pharmaceutical composition” refers to a chemical or biological composition suitable for administration to a mammal.

### Ion Exchange Resin Particles

The invention provides abuse-resistant oral drug dosage forms comprising at least two distinct populations of ion exchange resin complex particles, the first being drug-resin complex particles and the second being aversive agent-containing resin particles. Drugs are typically bound to the ion exchange resin particles by ionic bonds. In one embodiment, each of these respective populations of particles comprises at least one pharmacologically active drug or aversive agent bound to particles of an ion-exchange resin to provide a drug-resin complex or aversive agent-resin complex.

In one embodiment of the invention, the first population of drug-resin complex particles comprising an analgesically effective amount of the opioid drug is optionally coated with a water-permeable diffusion barrier coating that is insoluble in gastrointestinal fluids, thereby providing a controllable sustained release of opioid drug under conditions encountered in the gastrointestinal tract. In another embodiment of the invention, the first population of drug-resin complex particles comprising an analgesically effective amount of the opioid drug may also include an enteric coating, thereby allowing release of the opioid drug in the small intestine. In another embodiment of the invention, the diffusion barrier coating applied to the first population of drug-resin complex particles comprises an enteric coating.

In a non-limiting embodiment of the invention, the second population of aversive agent-containing resin particles comprising one or more aversive agents maintains these agents in a non-releasable or substantially non-releaseable form through the use of an ion exchange resin, which impedes the release of the agent(s) through ionic binding, in conjunction with a polymer coating, which substantially prevents release of the aversive agent under normal use conditions. Only when exposed to appropriate ionic conditions will the one or more aversive agents release from the ion exchange resin.

In one embodiment of the invention, the one or more aversive agents are opioid antagonists. In another embodiment of the invention, the one or more aversive agents may be mixed or combined with one or more opioid antagonists prior to loading onto the ion exchange resin. In another embodiment of the invention, the one or more aversive agents may be loaded onto the ion exchange resin, followed by loading and/or overlaying of one or more opioid antagonists, followed by application of a polymer coating which substantially prevents release of the aversive agent under normal use conditions. In yet another embodiment of the invention, one or more opioid antagonists may be loaded onto the ion exchange resin, followed by loading and/or overlaying of one or more aversive agents, followed by application of a polymer coating which substantially prevents release of the aversive agent under normal use conditions.

In a further embodiment of the invention, the composition comprising the drug-resin complex particles may also contain unbound drug or drugs bound by non-ionic means.

Ion-exchange resins suitable for use in the preparations and methods described herein are water-insoluble and comprise a pharmacologically inert organic and/or inorganic matrix containing covalently bound functional groups that are ionic or capable of being ionized under appropriate pH conditions. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix preferably comprises silica gel modified by the addition of ionic groups. Covalently bound ionic groups may be strongly acidic (e.g., sulfonic acid, phosphoric acid), weakly acidic (e.g., carboxylic acid), strongly basic (e.g., primary amine), weakly basic (e.g. quaternary ammonium), or a combination of acidic and basic groups.

In general, the types of ion-exchangers suitable for use in ion-exchange chromatography and for such applications as deionization of water are suitable for use in the controlled release of drug preparations. Suitable ion exchange resins are also sold under the trade names Amberlite and Dowex. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975), incorporated herein by reference. Exemplary ion-exchange resins that can be used in the present invention have exchange capacities below about 6 milliequivalents (meq)/gram and preferably below about 5.5 meq/gram.

Typically, the size of the ion-exchange particles is from about 30 microns to about 500 microns, preferably the particle size is within the range of about 40 microns to about 150 microns for liquid dosage forms, although particles up to about 1,000 microns can be used for solid dosage forms, e.g., tablets and capsules. Particle sizes substantially below the lower limit are difficult to handle in all steps of the processing. Commercially-available ion-exchange resins having an irregular shape and larger diameters up to about 200 microns, are gritty in liquid dosage forms and have a greater tendency to fracture when subjected to drying-hydrating cycles. Moreover, it is believed that the increased distance that a displacing ion must travel in its diffusion into these large particles, and the increased distance the displaced drug must travel in its diffusion out of these large particles, cause a measurable but not readily controlled prolongation of release, even when the drug-resin complexes are uncoated. Release of drug from uncoated drug-resin complexes with particle sizes in the approximate range of 40 microns to 150 microns is relatively rapid in the appropriate environment. Satisfactory control of the release of opioid drug from such complexes is achieved almost exclusively by the applied water-soluble diffusion barrier coating.

Both regularly and irregularly shaped particles may be used as resins. Regularly shaped particles are those particles that substantially conform to geometric shapes, such as spherical, elliptical, cylindrical and the like, which are exemplified by Dow XYS-40010.00 and Dow XYS-40013.00 (The Dow Chemical Company, Midland, Mich.). Irregularly shaped particles are all particles not considered to be regularly shaped, such as particles with amorphous shapes and particles with increased surface areas due to surface channels or distortions. Irregularly shaped ion-exchange resins of this type are exemplified by Amberlite IRP-69 (Rohm and Haas, Newark, Del.).

In a preferred embodiment of the invention, regularly shaped particles are utilized for the preparation of both populations of drug-resin complex particles and aversive agent-containing particles. In a preferred embodiment of the invention, both populations of particles are substantially similar in one or more of size, color, texture, and appearance. This is facilitated for example through the use of the same coating material for both populations of drug-resin complex particles, as discussed in greater detail below. In this embodiment, it is difficult for the potential drug abuser to visibly discern the population of opioid-harboring drug-resin complex particles from the population of aversive agent-containing particles. In a particularly preferred embodiment of the invention, both populations of particles are substantially similar in all of size, color, texture, and appearance.

Two of the preferred resins of this invention are Amberlite IRP-69 and Dow XYS-40010.00. Both are sulfonated polymers composed of polystyrene cross-linked with 8% of divinylbenzene, with an ion-exchange capacity of about 4.5 to 5.5 meq/g of dry resin (Na⁺-form). Their essential difference is in physical form. Amberlite IRP-69 consists of irregularly-shaped particles with a size range of 47 microns to 149 microns produced by milling the parent large-sized spheres of Amberlite IRP-120. The Dow XYS-40010.00 product consists of spherical particles with a size range of 45 microns to 150 microns. Another useful exchange resin, Dow XYS-40013.00, is a polymer composed of polystyrene cross-linked with 8% of divinylbenzene and functionalized with a quaternary ammonium group; its exchange capacity is normally within the range of approximately 3 to 4 meq/g of dry resin.

The following U.S. Patents and Patent Application Publications describe resins suitable for use in the preparations and methods described herein: U.S. Pat. Nos. 4,221,778; 4,996,047; and 5,980,882; U.S. Patent Application Publication Nos. 2003/0099711; 2006/0193877; 2007/0059270; 2007/01400983; 2007/0148239; and 2009/0011027. The disclosure of each of these patents and publications is incorporated herein by reference in their entireties.

As described herein, one of skill in the art may modify the resin particle size to modify a drug release profile and ultimately achieve a desired in vivo serum concentration profile.

### Chelating Agents

In one embodiment of the invention directed to the second population of aversive agent-resin particles, the aversive agent is further maintained in a non-releasable or substantially non-releaseable form through the inclusion of one or more chelating agents. The one or more chelating agents may be used in conjunction with the ion-exchange resin and a polymer coating. In this embodiment, the one or more chelating agents are preferably added following binding of the aversive agent to the ion-exchange resin.

In one embodiment, the one or more chelating agents are not covalently bound to the ion exchange resin complex. In another embodiment, the one or more chelating agents are covalently bound to the ion exchange resin complex. Neither resins nor aversive agent-resin complexes are soluble in water, and therefore the reactions are typically performed in suspension. The one or more chelating agents may be added to the liquid in which the resin is suspended. The aversive agent-resin complex may optionally be dried before adding the one or more chelating agents.

In another embodiment of the invention, the one or more chelating agents are administered between coating layers over the aversive agent-resin complex. In this embodiment, the aversive agent-resin complex is first overlayed with a suitable polymer coating. The one or more chelating agents are then applied to that polymer coating. Following application of the one or more chelating agents, another layer of suitable polymer coating is administered over the one or more chelating agents, thereby trapping or “sandwiching” the one or more chelating agents between layers of the appropriate polymer coating(s). The polymer coating layers may comprise the same polymer coating, or may comprise different polymer coatings. The invention also contemplates one or more “embedded” chelating agent layers in which one or more chelating agents are included in the coating solution at a high concentration, and are therefore present as part of the coating layer when applied to the aversive agent-resin complex particle. The invention further contemplates more than one “sandwiched” chelating agent layer (a sandwich layer comprising one or more chelating agents between two coating layers), such as for example 2, 3, or more sandwich layers. In one embodiment of the invention, the one or more “embedded” chelating agent layer(s) may be combined with one or more “sandwiched” chelating agent layers. In a preferred embodiment of the invention, the two polymer coating layers used to trap the one or more chelating agents comprise ethylcellulose at a coat weight of about 30% by weight of the aversive agent-resin complex particle, which is an amount sufficient to substantially prevent release of the aversive agent and the one or more chelating agents. The one or more chelating agents is preferably ethylenediaminetetraacetic acid (“EDTA”). This embodiment also contemplates the inclusion of an enteric coating applied over the outer coating layer.

In another embodiment of the invention, the aversive agent-resin complex is first overlayed with a suitable polymer coating. The one or more chelating agents are then applied to that polymer coating. Following application of the one or more chelating agents, another layer of suitable polymer coating is administered over the one or more chelating agents, thereby trapping or “sandwiching” the one or more chelating agents between layers of the appropriate polymer coating(s). In this embodiment of the invention, the inner or the outer coating layer may be an enteric coating layer. This embodiment contemplates one or more “embedded” chelating agent layers in which one or more chelating agents are included in the enteric and/or non-enteric coating solution at a high concentration, and are therefore present as part of the enteric or non-enteric coating layer when applied to the aversive agent-resin complex particle. The invention further contemplates more than one “sandwiched” chelating agent layer (a sandwich layer comprising one or more chelating agents between two coating layers), such as for example 2, 3, or more sandwich layers, wherein at least one of the coating layers is an enteric coating layer. In this embodiment, the one or more “embedded” chelating agent layer(s) may be combined with one or more “sandwiched” chelating agent layers.

Various chelating agents may be used with aversive agent-resin complex particles, alone or in combination with other chelating agents. In one embodiment of the invention, the chelating agent is EDTA or one or more salts thereof. Salts of EDTA include edetate calcium disodium, edetate trisodium, edetate disodium, and edetate sodium. In a preferred embodiment of the invention, the chelating agent is EDTA.

Other useful chelating agents include desferrioxamine B, deferoxamine, dithiocarb sodium, penicillamine, pentetate calcium, sodium salts of pentetic acid, succimer, trientine, nitrilotriacetic acid, trans-diaminocyclohexanetetraacetic acid (DCTA), diethylenetriaminepentaacetic acid, bis(aminoethyl)glycolether-N,N,N′,N′-tetraacetic acid, iminodiacetic acid, citric acid, tartaric acid, fumaric acid, or salts thereof. Preferably the chelating agent is completely non toxic and has no pharmacological effect on the body except for its chelating effect.

The chelating agent can be present in a concentration of from about 0.001 percent to about 10 percent by weight, more preferably from about 0.1 to about 5 percent by weight. In a preferred embodiment of the invention, the concentration of the chelating agent is about 0.3 to about 0.4 percent by weight for a solid dosage form. For a dosage form which is a suspension, the concentration of the chelating agent is most preferably about 0.05% by weight.

### Drugs

Drugs that are suitable for use in the oral dosage forms include, but are not limited to, hypnotics such as phenobarbital sodium; and opioid analgesic drugs such as alfentanil, allylprodine, alphaprodine, anileridine, benzylmorphine, bezitramide, buprenorphine, butorphanol, clonitazene, codeine, cyclazocine, desomorphine, dextromoramide, dezocine, diamorphone, diampromide, dihydrocodeine, dihydromorphine, dimenoxadol, dimepheptanol, dimethylthiambutene, dioxaphetyl butyrate, dipipanone, eptazocine, etorphine, dihydroetorphine, ethotheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium and compounds contained therein, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, salts thereof, mixtures of any of the foregoing, mixed mu-agonists/antagonists, mu-antagonist combinations, and the like.

Preferably, drugs which may be used in the oral dosage forms include drugs for the indication of moderate/severe pain such as oxycodone, morphine (sulfate), oxycodone/acetaminophen, hydromorphone, tramadol hydrochloride, hydrocodone/acetaminophen, and codeine.

Aversive agents that are suitable for use in the oral dosage forms may take the form of, for example, bittering agents, irritants, gelling agents, or opioid antagonists.

Opioid antagonists suitable for use in the oral dosage forms include, but are not limited to, naltrexone, nalmefene, cyclazacine, levallorphan, dextromethorphan ((+)-3 -hydroxy-N-methylmorphinan), its metabolite dextrorphan ((+)-3-hydroxy-N-methylmorphinan), amantadine (1-amino adamantine), memantine (3,5 dimethylaminoadamantone), d-methadone (d-form of 6-dimethylamino-4, 4-diphenyl-3-heptanone hydrochloride), naloxone, etorphine and dihydroetorphine, and mixtures thereof.

Bittering agents are intended to discourage abusers from tampering with the dosage form and subsequently inhaling or swallowing the tampered dosage form. The term “bittering agent” as used herein includes compounds used to impart a bitter taste or bitter flavor to a drug abuser following tampering with the dosage form.

Bittering agents that may be used in the oral dosage forms include, but are not limited to, one or more of the following non-limiting listing of bittering agents: natural, artificial and synthetic flavor oils and flavoring aromatics and/or oils, oleoresins and extracts derived from plants, leaves, flowers, fruits, and so forth, and combinations thereof. Flavor oils include, but are not limited to, spearmint oil, peppermint oil, eucalyptus oil, oil of nutmeg, allspice, mace, oil of bitter almonds, menthol and the like. Further useful bittering agents include, but are not limited to, artificial, natural and synthetic fruit flavors such as citrus oils including lemon, orange, lime, grapefruit, and fruit essences and so forth. Further useful bittering agents include, but are not limited to, sucrose derivatives (e.g., sucrose octaacetate), chlorosucrose derivatives, quinine sulphate, and the like. In one embodiment of the invention, the preferred bittering agent for use in the present invention is Denatonium Benzoate NF-Anhydrous, sold under the name Bitrex™ (Macfarlan Smith Limited, Edinburgh, UK). The invention particularly contemplates the use of ionic forms or derivatives of the bittering agents recited herein.

Bittering agents may be added to the form in an amount of less than about 50% by weight, preferably less than about 10% by weight, more preferably less than about 5% by weight of the dosage form, and still more preferably in an amount ranging from about 0.1 to 1.0 percent by weight of the dosage form depending on the particular bittering agent(s) used. A dosage form including a bittering agent preferably discourages improper usage of the tampered dosage form by imparting a disagreeable taste or flavor to the tampered dosage form.

Irritants and gelling agents are intended to discourage abusers from tampering with the dosage form and subsequently inhaling, injecting, or swallowing the tampered dosage form. The term “irritant” as used herein includes compounds used to impart burning, irritating, or uncomfortable sensations to a drug abuser following tampering with the dosage form. The term “gelling agent” as used herein includes compounds used to impart gel-like or thickening qualities to the form following tampering with the dosage form.

Irritants that may be used with the oral dosage forms include, but are not limited to, one or more of the following non-limiting listing of irritants: capsaicin (trans 8-methyl-N-vanillyl-6-noneamide), a capsaicin analog with similar properties as capsaicin, and the like. Representative capsaicin analogues include, but are not limited to, resiniferatoxin, tinyatoxin, heptanoylisobutylamide, heptanoyl guaiacylamide, other isobutylamides or guaiacylamides, dihydrocapsaicin, homovanillyl octylester, nonanoyl vanillylamide, or other compounds of the class known as vanilloids. See for example U.S. Pat. No. 5,290,816 (resiniferatoxin); U.S. Pat. No. 4,812,446 (capsaicin analogs); and Ton et al., British Journal of Pharmacology, 10, pp. 175 182 (1955) discussing pharmacological actions of capsaicin and its analogs.

Suitable irritants such as capsaicin or analogues thereof may be included at a concentration of between about 0.00125% and about 50% by weight, preferably between about 1 and about 7.5% by weight, and most preferably between about 1 and about 5% by weight.

Gelling agents that may be used with the oral dosage forms include, but are not limited to, one or more of the following non-limiting listing of gelling agents: sugars or sugar derived alcohols, such as mannitol, sorbitol, and the like, starch and starch derivatives, cellulose derivatives, such as microcrystalline cellulose, sodium carboxymethyl cellulose, methylcellulose, ethyl cellulose, polymethacrylates, hydroxyethyl cellulose, hydroxypropyl cellulose, and hydroxypropyl methylcellulose, attapulgites, bentonites, dextrins, alginates, carrageenan, gum tragacant, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof, etc. In a preferred embodiment, the gelling agent is xanthan gum or pectin. Pectin useful with the invention includes, but is not limited to, purified or isolated pectates as well as crude natural pectin sources such as apple, citrus or sugar beet residues which have been subjected, when necessary, to esterification or de-esterification, e.g., by alkali or enzymes. In a preferred embodiment, the pectins used in the invention are derived from citrus fruits such as lime, lemon, grapefruit, and orange. The invention particularly contemplates the use of ionic forms or derivatives of the gelling agents recited herein.

Gelling agents may be added to the form in a ratio of gelling agent to opioid drug of from about 1:40 to about 40:1 by weight, preferably from about 1:1 to about 30:1 by weight, and more preferably from about 2:1 to about 10:1 by weight of the opioid drug.

In further embodiments of the invention, the dosage form may include a bittering agent; an irritant; a gelling agent; an opioid antagonist; a bittering agent and an irritant; a bittering agent and a gelling agent; a bittering agent and an opioid antagonist; an irritant and a gelling agent; an irritant and an opioid antagonist; an opioid antagonist and a gelling agent; a bittering agent, irritant and a gelling agent; a bittering agent, irritant, and an opioid antagonist; an opioid antagonist, irritant and a gelling agent; and an opioid antagonist, bittering agent, irritant and a gelling agent.

In one embodiment, the dosage form generates a viscous gel after tampering when dissolved in an aqueous liquid (from about 0.5 to about 10 ml and preferably from 1 to about 5 ml), causing the resulting mixture to have a viscosity of at least about 10 cP, more preferably a viscosity of at least about 60 cP. In another embodiment, the dosage form generates a viscous gel after tampering when dissolved in an aqueous liquid (from about 0.5 to about 10 ml and preferably from 1 to about 5 ml) and then heated (e.g., greater than about 45° C.), causing the resulting mixture to have a viscosity of at least about 10 cP, more preferably a viscosity of at least about 60 cP.

In a preferred embodiment of the invention, opioid drug and aversive agent combinations which may be used comprise, or alternatively consist of, one or more of the opioid drugs set forth herein in combination with one or more of the opioid antagonists set forth herein.

In one embodiment, the one or more aversive agents are bound to the ion exchange resin. In another embodiment of the invention, the one or more aversive agents are administered between coating layers over the ion exchange-resin particles. In this embodiment, the ion exchange-resin particle is first overlayed with a suitable polymer coating. The one or more aversive agents are then applied to that polymer coating. Following application of the one or more aversive agents, another layer of suitable polymer coating is administered over the one or more aversive agents, thereby trapping or “sandwiching” the one or more aversive agents between layers of the appropriate polymer coating(s). The invention also contemplates one or more “embedded” aversive agent layers in which one or more aversive agents are included in the coating solution at a high concentration, and are therefore present as part of the coating layer when applied to the aversive agent-resin complex particle. In one embodiment of the invention, the one or more “embedded” aversive agent layer(s) may be combined with one or more “sandwiched” aversive agent layers. In some embodiments, the layer of the sandwich coating nearest to the ion exchange resin comprises a water soluble polymer.

In a related embodiment, the ion exchange-resin particle does not contain bound aversive agent prior to coating with a “sandwich” layer of one or more aversive agents. The polymer coating layers may comprise the same polymer coating, or may comprise different polymer coatings. The invention also contemplates more than one “sandwiched” aversive agent layer (a sandwich layer comprising one or more aversive agents between two coating layers), such as for example 2, 3, or more sandwich layers. In a preferred embodiment of the invention, the two polymer coating layers used to trap the one or more aversive agents comprise ethylcellulose at a sufficient concentration to substantially prevent release of the aversive agent. Any of these embodiments also contemplate the inclusion of an enteric coating applied over the outer coating layer.

In another embodiment of the invention, the one or more aversive agents are bound to the ion exchange resin. The one or more aversive agents are then coated with a water-soluble coating. Following application of the water-soluble coating, an aversive agent is applied over the water-soluble coating. In this embodiment of the invention, the aversive agent has a higher affinity for the ion exchange resin than the bound drug. A layer of a suitable water insoluble, permeable polymer coating is administered over the one or more aversive agents, thereby trapping or “sandwiching” the one or more aversive agents between the coating layers. In a preferred embodiment of the invention, the outer coating layer is an ethylcellulose coating. Following ingestion, the ion exchange resin particle becomes hydrated. On hydration, the water-soluble inner coating becomes porous or solubilizes, and the aversive agent displaces the bound drug due to its higher affinity for the ion exchange resin. The liberated drug is free to pass through the water insoluble, permeable coating while the aversive agent remains substantially bound to the ion exchange resin.

In another embodiment, the one or more polymer coating layers comprise one or more aversive agents contained within said one or more polymer coating layers.

In another embodiment of the invention, the compositions of this invention may optionally contain one or more other known therapeutic agents. The following U.S. Patents and Patent Application Publications describe additional drugs suitable for use in the preparations and methods described herein: U.S. Pat. Nos. 4,221,778; 4,619,934; 4,996,047; and 5,980,882; U.S. Publication Nos. 2003/0099711; 2006/0193877; 2007/0059270; 2007/01400983; 2007/0148239; and 2009/0011027. The disclosure of each of these patents and publications is incorporated herein by reference in their entireties.

### Preparing Ion Exchange-Resin Complexes

Binding of opioid drug or aversive agent to the ion exchange resin can be accomplished using methods known in the art. Indeed, one of ordinary skill in the art can easily determine the appropriate method depending on the opioid drug and/or aversive agent. Typically four general reactions are used for a basic opioid drug and/or aversive agent, these are: (a) resin (Na⁺-form) plus opioid drug or aversive agent (salt form); (b) resin (Na⁺-form) plus opioid drug or aversive agent (as free base); (c) resin (H³⁰ -form) plus opioid drug or aversive agent (salt form); and (d) resin (H³⁰ -form) plus opioid drug or aversive agent (as free base). All of these reactions except (d) have cationic by-products and these by-products, by competing with the cationic opioid drug or aversive agent for binding sites on the resin, reduce the amount of opioid drug or aversive agent bound at equilibrium. For basic opioid drug or aversive agent, stoichiometric binding of opioid drug or aversive agent to resin is accomplished only through reaction (d). Without being limited by theory, it is believed that the extent of opioid drug binding is critical to the maintenance of the integrity of the diffusion barrier coating.

Four analogous binding reactions can be carried out for binding an acidic opioid drug or aversive agent to an anion exchange resin. These are: (a) resin (Cl⁻-form) plus opioid drug or aversive agent (salt form); (b) resin (Cl⁻-form) plus opioid drug or aversive agent (as free acid); (c) resin (OH⁻-form) plus opioid drug or aversive agent (salt form); and (d) resin (OH⁻-form) plus opioid drug or aversive agent (as free acid). All of these reactions except (d) have ionic by-products and the anions generated when the reactions occur compete with the anionic opioid drug or aversive agent for binding sites on the resin with the result that reduced levels of opioid drug or aversive agent are bound at equilibrium. For acidic opioid drug or aversive agent, stoichiometric binding of opioid drug or aversive agent to resin is accomplished only through reaction (d).

The binding may be performed, for example, as a batch or column process, as is known in the art. The opioid drug or aversive agent-resin complexes may be prepared by a batch process that is based on reaction (d). The drug or aversive agent to be loaded on the ion exchange resin beads may be dissolved in an aqueous medium or in a solvent miscible with water to make a solution. The opioid drug or aversive agent-containing solution is then placed in a slurry of the resin or a column loaded with resin. The opioid drug or aversive agent-resin complex thus formed is collected by filtration and washed with deionized or purified water to ensure removal of any unbound opioid drug or aversive agent. The complexes are usually air-dried in trays or fluid bed dried at about 25° C. to about 60° C.

The amount of opioid drug or aversive agent that can be loaded onto a resin will typically range from about 1% to about 80%, preferably about 15% to about 60%, by weight of the loaded opioid drug or aversive agent resin particles. A skilled artisan with little or no experimentation can readily determine the optimum loading for any opioid drug or aversive agent-resin complex. In a preferred embodiment, loadings of about 30% to about 60% by weight of the opioid drug or aversive agent-resin particles can be employed. In a preferred embodiment of the invention, the aversive agent and/or opioid drug is underloaded (e.g., about 15% to about 50% by weight of particles).

The following U.S. Patents and Patent Application Publications describe ion exchange resin complexes suitable for use in the preparations and methods described herein: U.S. Pat. Nos. 4,221,778; 4,996,047; and 5,980,882; U.S. Publication Nos. 2003/0099711; 2006/0193877; 2007/0059270; 2007/01400983; 2007/0148239; and 2009/0011027. The disclosure of each of these patents and publications is incorporated herein by reference in their entireties.

The preferred dosage of opioid drug can range from about 1 mg per 70 kg body weight of subject to about 800 mg per 70 kg body weight per unit dose. Preferably, the dosage of opioid drug is from about 5 mg per 70 kg body weight to about 600 mg per 70 kg body weight in the unit dosage form. In a more preferred embodiment, the dosage of opioid drug is from about 10 mg per 70 kg body weight to about 200 mg per 70 kg body weight in the unit dosage form. The invention also contemplates equianalgesic doses of opioid drugs, such as for example oxycodone, codeine, hydrocodone, hydromorphone, levorphanol, meperidine, methadone, and/or morphine. Equianalgesic doses of opioid drugs are set forth in Table 1 of U.S. Pat. No. 6,696,088, which is incorporated herein by reference.

The preferred dosage of aversive agent, such as for example an opioid antagonist, can range from about 1 mg per 70 kg body weight of subject to about 800 mg per 70 kg body weight per unit dose. Preferably, the dosage of aversive agent is from about 10 mg per 70 kg body weight to about 500 mg per 70 kg body weight in the unit dosage form.

In one embodiment, the ratio of the opioid drug and the aversive agent may be from about 1:1 to about 90:1 by weight, preferably about 1:1 to about 20:1 by weight, or still more preferably about 15:1 to about 30:1 by weight. Another preferred ratio of the opioid drug and the aversive agent may be from about 1:1 to about 10:1 by weight. The weight ratio of the opioid drug to aversive agent refers to the weight of the active ingredients. For example, the weight of the aversive agent excludes the weight of the coating or matrix that renders the aversive agent substantially non-releasable. In a preferred embodiment of the invention, the opioid drug comprises oxycodone or hydrocodone and is present in the amount of about 15-45 mg and the aversive agent comprises naltrexone and is present in about 0.5-5 mg, or a ratio of opioid drug to aversive agent of about 3:1 to about 90:1.

In one embodiment of the invention, the dosage forms do not release, or do not substantially release, the aversive agent when administered intact, such that the ratio of the amount of aversive agent released from the dosage form after tampering to the amount of the aversive agent released from the intact dosage form is about 4:1 or greater, based on the in-vitro dissolution at 1 hour of the dosage form in 900 ml of Simulated Gastric Fluid using a USP Type II (paddle) apparatus at 75 rpm at 37° C.

In another embodiment of the invention, the intact dosage forms release a small amount of aversive agent at 1 hour following oral administration, i.e., the dosage forms release no more than about 0.025 mg of aversive agent after 1 hour. In these embodiments, the dosage forms preferably do not release 0.25 mg or more of aversive agent at 1 hour following oral administration. In a preferred embodiment, the intact dosage forms do not release more than about 2% by weight of the aversive agent following administration.

As described herein, one of skill in the art may increase or decrease the amount of opioid drugs or aversive agents loaded on a resin particle to modify a release profile and ultimately achieve a desired in vivo serum concentration profile.

### Impregnation

In some embodiments of the invention, one or both populations of resin complex particles described herein can be impregnated with a humectant substantially as described in U.S. Pat. No. 4,221,778. The humectant can be added as an ingredient in the resin-drug complexation or resin-aversive agent complexation step or preferably, the particles can be treated with the humectant after complexing. This treatment helps particles retain their geometry, and enables the effective application of barrier coatings, such as diffusion barrier coatings, to such particles. One preferred humectant is polyethylene glycol, a hydrophilic agent. Other effective humectant agents include, for example, propylene glycol, lactose, methylcellulose, hydroxypropylmethylcellulose, sugar alcohols such as sorbitol, mannitol, polyvinylpyrrolidone, carboxypolymethylene, xanthan gum, propylene glycol, alginate and combinations of these agents. The humectant may be added in an amount of up to about 50 parts by weight of the resin or 50 to 150 parts per 100 parts of the resin by weight; such humectant levels have been found to be effective. Preferably, the humectant (solvating agent) is added in an amount of about 75 to about 100 parts of resin. Such pretreatment of drug-resin complex enables the effective use of diffusion barrier coatings, resulting in the ability to effectively prolong the release of drugs from drug-resin complexes.

### Coatings

Next, both populations of resin particles may be coated with one or more coatings. In one embodiment of the invention, the coatings applied to the first population of drug-resin complex particles comprise the same materials, but have coatings of differing weights for the respective populations of particles. In this embodiment of the invention, the first population of drug-resin complex particles comprises a diffusion barrier coating having a coat weight of about 5% to about 60% based on the total weight of the coated drug-resin complex particles in order to obtain a desired sustained release profile, while the second population of aversive agent-resin complex particles comprises a polymeric coating having a coat weight of about 20% to about 90% based on the total weight of the coated aversive agent-resin complex particles in order to substantially prevent the release of the one or more aversive agents. In a preferred embodiment of the invention, the percent coat weight applied to the aversive agent-containing particles is greater than or equal to the percent coat weight applied to the drug-resin complex particles, more preferably a ratio of about 5:1 applied to aversive agent-containing particles and drug-resin complex particles, respectively.

In another embodiment of the invention, the two populations of drug-resin particles and aversive agent-resin particles comprise coatings comprising different polymeric materials. For example, a water-permeable, film-forming polymer diffusion barrier coating may be applied to the first population of opioid-containing drug-resin complex particles. The coating applied to the second population of aversive agent-containing resin complex particles comprises a polymeric coating designed to substantially or completely inhibit release of the aversive agent into the subject (e.g., sequestering) under normal use conditions. In this embodiment of the invention, the first population of drug-resin complex particles comprises a diffusion barrier coating having a coat weight of about 5% to about 60% of the drug-resin complex particles in order to obtain a desired sustained release profile, while the second population of aversive agent-resin complex particles comprises a polymeric coating having a coat weight of about 20% to about 90% in order to substantially prevent the release of the one or more aversive agents. It is understood that the coat weight percentages may change based on the type of polymer applied to the particles.

The invention contemplates the application of the same coating material to attain two populations of drug-resin and aversive agent-resin particles substantially similar in one or more of appearance, color, texture, and/or size.

As explained in more detail below, one of ordinary skill in the art appreciates from this disclosure that the purpose of these coatings is different for the respective populations of drug-resin and aversive agent-resin complex particles. Any coating procedure which provides a contiguous coating on each resin complex particle without significant agglomeration of particles may be used. Coatings may be applied with a fluid-bed coating apparatus having the Wurster coater configuration. Measurements of particle size distribution can be done before and after coating to show that agglomeration of particles is insignificant.

### I. Opioid-Containing Drug-Resin Complex Particles

The polymer coating applied to the first population of opioid-containing drug-resin complex particles may be a diffusion barrier coating intended to provide a sustained release of said opioid drug following oral administration. With respect to the first population of opioid-containing drug-resin complex particles, the polymer coating may be any of a large number of natural or synthetic film-formers used singly, or in admixture with each other, and optionally in admixture with plasticizers, pigments and other substances to alter the characteristics of the coating. Such a coating should be insoluble in and permeable to water. The water-permeable barrier comprises a pharmaceutically acceptable polymer such as, for example, ethylcellulose, methylcellulose, hydroxypropylmethylcellulose (HPMC), hydroxyethlycellulose (HEC), acrylic acid ester, cellulose acetate phthalate, HEC phthalate, HPMC phthalate or other cellulosic polymers, or mixtures of polymers. Additional examples of coating polymers are described by R. C. Rowe in Materials Used in Pharmaceutical Formulation (A. T. Florence, editor), Blackwell Scientific Publications, Oxford, 1-36 (1984), incorporated herein by reference. Preferably the diffusion barrier coating is ethyl cellulose, for example, an ethyl cellulose having the content of ethoxyl group from 44 to 47.5%, preferably from 45 to 46.5%.

One commercially available aqueous dispersion of ethylcellulose is Aquacoat® (FMC Corp., Philadelphia, Pa., U.S.A.). Aquacoat® is typically prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is preferable to intimately mix the Aquacoat® with a suitable plasticizer prior to use.

Another aqueous dispersion of ethylcellulose is commercially available as Surelease® (Colorcon, Inc., West Point, Pa., U.S.A.). This product is typically prepared by incorporating plasticizer into the dispersion during the manufacturing process. A hot melt of a polymer, plasticizer (e.g., dibutyl sebacate), and stabilizer (e.g., oleic acid) may be prepared as a homogeneous mixture, which is then diluted with an alkaline solution to obtain an aqueous dispersion which can be applied directly onto substrates.

Another alternative coating material is a mixture of an insoluble film forming polymer and a water soluble pore former or polymer. One preferred water soluble polymer is methyl cellulose.

The barrier coating materials can be applied as a solvent-based solution or as an aqueous based suspension. Optimum coat weight and coat thickness may be determined for each drug-resin complex and generally depend on the drug release characteristics of the resin for a particular drug. For example, for drug release times within about 1 hour to about 4 hours, the drug-resin complex may be coated with a light coat weight. A light coat weight is a coat weight present in the amount of about 5% to about 20% by weight of the drug-resin complex, depending on the nature of the coating used. For certain coatings (i.e., ethylcellulose), drug release times from about 6 hours to 10 hours, a medium coat weight may be used, e.g., a coat weight present in the amount of 10% to about 40% by weight of the drug-resin complex, depending on the nature of the coating used. For drug release times for about 12 hours, a heavy coat weight may be used, e.g., a coat weight of about 20% to 60% by weight of the drug-resin complex, depending on the nature of the coating used.

The following U.S. Patents and Patent Application Publications describe coating materials suitable for use in the preparations and methods described herein: U.S. Pat. Nos. 4,221,778; 4,996,047; and 5,980,882; U.S. Publication Nos. 2003/0099711; 2006/0193877; 2007/0059270; 2007/0140983; 2007/0148239; and 2009/0011027. The disclosure of each of these patents and publications is incorporated herein by reference in their entireties.

As noted previously and as described herein, one of skill in the art may increase or decrease the amount of coating, or change the composition of the coating, applied to a resin particle to modify a drug release profile and ultimately achieve a desired in vivo serum concentration profile.

### II. Aversive Agent-Containing Resin Complex Particles

The polymer coating applied to the second population of aversive agent-containing resin complex particles is intended to sequester or inhibit release, or substantially inhibit release, of the aversive agent under normal use conditions. In a preferred embodiment, the polymer coating inhibits or substantially inhibits release of an aversive agent in an amount sufficient to at least partially or substantially deny the drug abuser an euphoric effect under normal use conditions. In this preferred embodiment, less than about 5% by weight of the aversive agent is released from the finally formulated dosage form under normal use conditions after the finally formulated dosage form containing both opioid drug and aversive agent is orally administered. In a particularly preferred embodiment, there is no significant release of the aversive agent from the finally formulated dosage form under normal use conditions after the finally formulated dosage form containing both opioid drug and aversive agent is orally administered. More preferably, the threshold of release does not exceed FDA levels for a given drug.

In another preferred embodiment, the amount released from an intact dosage form is 36% or less, preferably 25% or less, still preferably 10% or less, and still more preferably 6% or less of the antagonist after 36 hours based on the in-vitro dissolution of the dosage form in 900 ml of Simulated Gastric Fluid using a USP Type II (paddle) apparatus at 75 rpm at 37° C., with a switch to Simulated Intestinal Fluid at 1 hour.

Methods of producing non-releasable or substantially non-releasable coatings are known in the art, for example, as taught in U.S. Pat. Nos. 6,696,088 and 7,842,311, the disclosures of each of which are incorporated herein by reference in their entireties. For example, aversive agent-containing particles may be coated with a coating comprising hydrophobic materials(s), such as for example one or more cellulose polymers selected from ethylcellulose, cellulose acetate, cellulose propionate (lower, medium or higher molecular weight), cellulose acetate propionate, cellulose acetate butyrate, cellulose acetate phthalate and cellulose triacetate. In a preferred embodiment, the coating is ethylcellulose comprising an ethoxy content of between about 44 to about 60%. In one embodiment, ethylcellulose may be applied in the form of an organic solution.

In another embodiment, the non-releasable or substantially non-releasable coating comprises hydrophobic materials such as polylactic acid, polyglycolic acid, a co-polymer of polylactic and polyglycolic acid (“PLGA”), a polyanhydride, a polyorthoester, polycaprolactones, polyphosphazenes, polysaccharides, proteinaceous polymers, polyesthers, polydioxanone, polygluconate, polylactic-acid-polyethylene oxide copolymers, poly(hydroxybutyrate), polyphosphoesther or mixtures or blends of any of these.

In one embodiment, a biodegradable polymer comprises a PLGA having a molecular weight of about 2,000 to about 500,000 daltons. The ratio of lactic acid to glycolic acid is from about 100:0 to about 25:75, with the ratio of lactic acid to glycolic acid of 65:35 being preferred. PLGA may be prepared by the procedure set forth in U.S. Pat. No. 4,293,539 (Ludwig et al.), the disclosure of which is herein incorporated by reference in its entirety.

In another embodiment, the hydrophobic material may comprise a cellulose polymer selected from a cellulose ether, a cellulose ester, a cellulose ester ether, and cellulose. As taught in U.S. Pat. Nos. 6,696,088 and 7,842,311, cellulosic polymers have a degree of substitution (“D.S.”) on the anhydroglucose unit, from greater than zero and up to and including 3. By degree of substitution is meant the average number of hydroxyl groups present on the anhydroglucose unit comprising the cellulose polymer that are replaced by a substituting group. Representative materials include a polymer selected from the group consisting of cellulose acylate, cellulose diacylate, cellulose triacylate, cellulose acetate, cellulose diacetate, cellulose triacetate, mono, di, and tricellulose alkanylates, mono, di, and tricellulose aroylates, and mono, di, and tricellulose alkenylates. Exemplary polymers include cellulose acetate having a D.S. and an acetyl content up to about 21%; cellulose acetate having an acetyl content up to about 32 to about 39.8%; cellulose acetate having a D.S. of about 1 to about 2 and an acetyl content of about 21 to about 35%; cellulose acetate having a D.S. of about 2 to about 3 and an acetyl content of about 35 to about 44.8%.

In another embodiment, the acrylic polymer is selected from acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate co-polymers.

The thickness of the coating will depend on the characteristics of the particular coating composition being used. However, it is well within the ability of one skilled in the art to determine by routine experimentation the optimum thickness of a particular coating required for a particular dosage form of the present invention. In one embodiment of the invention, the coating applied to the second population of aversive agent-containing resin particles has a coat weight present in the amount of about 20% to about 90% by weight of the aversive agent-containing resin particles, depending on the nature of the coating used. It is understood that these coat weights may be increased or decreased if necessary in order to achieve the desired sequestration or release-inhibition profile.

### III. Enteric Coatings

Another embodiment of the present invention is directed to providing an enteric coating on the first population of drug-resin particles. As is known in the art, an enteric coating is intended to prevent the active ingredients in the preparation, or dosage form, from disintegrating in the stomach, and to allow the active ingredient(s) to be released once the dosage form has passed into the small intestinal tract. Thus, polymeric materials that are suitable for enteric coating applications should be insoluble in a low pH medium typically having a value less than 3.5, but soluble in a higher pH medium typically having a value greater than 5.5. Thus, the objectives for using enteric coating materials in pharmaceutical dosage forms include: (a) to protect the stomach from the harmful effect(s) of an active ingredient, (b) to protect the active ingredient from the adverse effect(s) of gastric fluid, (c) to deliver an active ingredient to a particular region of the intestine, and (d) to provide a sustained release dosage form to the gastrointestinal tract.

Polymers that are commonly used as enteric coatings in pharmaceutical preparations include cellulosic materials such as cellulose acetate phthalate (C-A-P), cellulose acetate trimellitate (C-A-T), cellulose acetate succinate (C-A-S), hydroxypropyl methyl cellulose phthalate (HPMCP), hydroxypropyl methyl cellulose acetate succinate (HPMCAS) and carboxy methyl ethyl cellulose (CMEC). Other, non-cellulosic, polymers that are used as enteric coatings include copolymers of methacrylic acid and methyl methacrylate or ethyl acrylate, terpolymers of methacrylic acid, methacrylate, and ethyl acrylate, and polyvinyl acetate phthalate (PVAP).

The enteric coating can be present in amounts from about 10% to about 90% by weight based on the particle being coated. Preferably, the enteric coating is present in an amount from about 30% to about 60% by weight of the particle being coated.

In one embodiment of the invention, an enteric coating on some drug-resin containing particles is used in conjunction with an ethylcellulose coating on other drug-resin containing particles to provide an immediate release of the opioid drug in the stomach, and a sustained release of the opioid drug in subsequent portions of the gastrointestinal tract.

### IV. Plasticizers

The coatings set forth above may further include an effective amount of a plasticizer to improve the physical properties of the film(s). For example, because ethylcellulose has a relatively high glass transition temperature and does not form flexible films under normal coating conditions, it may be necessary to plasticize the ethylcellulose before using the same as a coating material. Generally, the amount of plasticizer included in a coating solution is based on the concentration of the film-former, e.g., most often from about 1 to about 50 percent by weight of the film-former. The concentration of the plasticizer, however, can only be properly determined after careful experimentation with the particular coating solution and method of application, which is within the abilities of the skilled artisan.

Examples of suitable plasticizers for ethylcellulose include water insoluble plasticizers such a dibutyl sebacate, diethyl phthalate, tributyl citrate and triacetin, although it is possible that other water-insoluble plasticizers (such as acetylated monoglycerides, phthalate esters, castor oil, etc.), or water-soluble plasticizers may be used. A plasticizer such as Durkex 500 vegetable oil may also be incorporated to improve the film forming property. In one alternative, it is desirable to incorporate a water-soluble substance, such as methyl cellulose, to alter the permeability of the coating.

### Dosage Forms

In one embodiment of the invention, the two populations of resin particles, the first comprising the opioid drug and the second comprising a substantially non-releasable form of an aversive agent, may be combined to produce oral liquid drug suspensions or forms. In another embodiment of the invention, the two populations of resin particles may also be combined in any of a suitable number of oral forms, such as for example in tablet forms, capsule forms; orally disintegrating tablet (ODT) forms such as fast dissolving discs; and sprinkle forms. The opioid drug-containing component of the forms is prepared as a controlled or sustained release oral form and mixed with an aversive agent-containing component. Populations of particles may be mixed in V-blenders, ribbon blenders, etc., then formulated into final products.

Liquid Drug Suspensions

Liquid oral dosage forms include aqueous and nonaqueous solutions, emulsions, suspensions, and solutions and/or suspensions reconstituted from non-effervescent granules, containing suitable solvents, preservatives, emulsifying agents, suspending agents, diluents, sweeteners, coloring agents, and flavoring agents. Liquid forms, such as syrups and suspensions, preferably contain from about 1% to about 50%, and more preferably from about 1% to about 25%, and most preferably from about 3% to about 10%, of the drug-resin complex. Other optional ingredients well known to the pharmacist's art may also be included in amounts generally known for these ingredients, for example, natural or artificial sweeteners, flavoring agents, colorants and the like to provide a palatable and pleasant looking final product; acidulants, for example, citric acid, ascorbic acid, or malic acid and the like to adjust pH; antioxidants, for example, butylated hydroxy anisole or butylated hydroxy toluene; and preservatives, for example, methyl or propyl paraben or sodium benzoate, to prolong and enhance shelf life.

In preparing the liquid oral dosage forms, the coated drug-resin and aversive agent-containing particles are incorporated into an aqueous-based orally acceptable pharmaceutical carrier consistent with conventional pharmaceutical practices. An “aqueous-based orally acceptable pharmaceutical carrier” is one wherein the entire or predominant solvent content is water. Typical carriers include simple aqueous solutions, syrups, dispersions and suspensions, and aqueous based emulsions such as the oil-in-water type. Preferably, the carrier is a suspension of the pharmaceutical composition in an aqueous vehicle containing a suitable suspending agent. Suitable suspending agents include Avicel RC-591 (a microcrystalline cellulose/sodium carboxymethyl cellulose mixture available from FMC), guar gum and the like. Such suspending agents are well known to those skilled in the art. While the amount of water in the compositions of this invention can vary over quite a wide range depending upon the total weight and volume of the drug-resin and aversive agent-containing particles and other optional non-active ingredients, the total water content, based on the weight of the final composition, will generally range from about 20 to about 75%, and, preferably, from about 20 to about 40%, by weight/volume.

Although water itself may make up the entire carrier, typical liquid forms may contain a co-solvent, for example, propylene glycol, glycerin, sorbitol solution and the like, to assist solubilization and incorporation of water-insoluble ingredients, such as flavoring oils and the like, into the composition. In general, therefore, the compositions of this invention preferably contain from about 5 to about 25 volume/volume percent and, most preferably, from about 10 to about 20 volume/volume percent, of the co-solvent.

As used herein, unless otherwise defined, the term “substantially free of organic solvent” means that the composition has less than 5% by weight of organic solvents, preferably, less than 2% by weight of the composition. More preferably, the term “substantially free of organic solvent” means that the composition has less than 1% by weight of organic solvents. Organic solvents include, but are not limited to, chloroform, methylene chloride, acetone, tetrahydrofuran, ethanol, methanol, and the like.

A. Specific Gravity

The specific gravity of the liquid drug suspension may also be altered as deemed necessary by one of ordinary skill in the art. Methods of altering the specific gravity of a liquid suspension are known in the art. For example, U.S. Patent Application Publication No. 2009/0176884, which is incorporated herein by reference in its entirety, describes a method of preparing liquid suspensions comprising suspending at least one pharmaceutically active compound in a suspending system (e.g., based on a thixotropic system) and matching/equilibrating the true density of the resin containing at least one pharmaceutical active with the specific gravity of the aqueous medium via a density adjusting agent. Accordingly, any of the methods described herein may further comprise a step of suspending drug-resin and aversive agent-containing particles in a suspending system to alter the specific gravity of the liquid drug suspension.

The liquid drug suspension may be manufactured using techniques known in the art such as those described in U.S. Patent Application Publication No. 2006/0193877, which is incorporated herein by reference in its entirety. Moreover, as described herein, one of skill in the art may modify the drug suspension to modify a drug release profile and ultimately achieve a desired in vivo serum concentration profile.

B. Methods of Preparing Suspensions with Desired Release Profiles

In another embodiment, the invention also provides for a method of formulating liquid drug suspensions having immediate release characteristics and sustained release characteristics. In this embodiment, the invention provides for a method of formulating a liquid drug suspension comprising suspending both populations of resin particles in a liquid suspension along with a third population of drug-resin complex particles. This form comprises the first population of opioid drug-resin complex particles comprising a water-permeable coating, the second population of aversive agent-containing drug-resin complex particles, and a third population of uncoated opioid drug-containing resin particles containing the same opioid drug as the first population of opioid drug-resin complex particles. In a preferred embodiment, the liquid drug suspension provides an early onset of therapeutic value, while masking the taste of the drug.

The invention may provide an early onset of therapeutic value in which at least 15% more of the opioid drug will be released within the first 15, 30, 60, 75, or 90 minutes of administration, as compared to conventional forms of the same opioid drug. In a preferred embodiment of the invention, substantially all of the early release of opioid drug will occur in the gastric space, with only an insignificant amount of opioid drug released in the oral cavity, in order to mask the taste of the drug.

The coating may be any coating described herein. A preferred coating is ethylcellulose. The opioid drug may be any of the opioid drugs described herein. It is understood that the various aspects described in this and other sections may be combined (e.g., the methods may use any of the dissolution media described herein, etc.).

### Methods of Preparing Tablets, Orally Disintegrating Tablets (ODTs) and Capsules

Methods of preparing tablet forms are well known in the art. In one embodiment of the invention, a tablet may be made by compression or molding, optionally with one or more accessory ingredients. Compressed tablets may be prepared by compressing, in a suitable machine, the therapeutic ingredient(s) in a free-flowing form such as a powder or granules, optionally mixed with a binder, lubricant, inert diluent, preservative, surface therapeutic or dispersing agent. Molded tablets may be made by molding, in a suitable machine, a mixture of the powdered compound moistened with an inert liquid diluent. The tablets may be optionally coated or scored and may be formulated so as to provide a slow or controlled release of the therapeutic ingredient therein.

The tablet may be made in any manner, and a variety of tableting methods are known in the art. Conventional methods for tablet production include direct compression (“dry blending”), dry granulation followed by compression, and wet granulation followed by drying and compression. Other methods include the use of compacting roller technology, e.g., a chilsonator (e.g., a dry granulation/roll compactor roller press system), drop roller, molding, casting, or extrusion technologies. All of these methods are well known in the art. The tablets may be formed by the direct compression method, which involves directly compacting a blend of the active ingredient, the excipient in the form of a hydrate, the water-swellable excipient, and any other appropriate optional ingredients. After blending, a pre-determined volume of particles are filled into a die cavity of a rotary tablet press, which continuously rotates as part of a “die table” from the filling position to a compaction position. The particles are compacted between an upper punch and a lower punch to an ejection position, at which the resulting tablet is pushed from the die cavity by the lower punch and guided to an ejection chute by a stationary “take-off” bar.

Orally disintegrating tablets (ODTs) and methods of producing ODTs such as tablets are known in the art, for example as taught in U.S. Pat. Nos. 5,464,632; 7,229,641; 7,390,503; and 7,425,341. The disclosure of each of these patents is incorporated by reference herein in their entireties.

Methods of producing hard and soft capsules such as gelatin capsules are well known in the art. For example, the biologically active compounds can be processed with pharmaceutically inert, inorganic or organic carriers for the production of pharmaceutical compositions. Lactose, corn starch or derivatives thereof, talc, stearic acid or its salts and the like can be used, for example, as such carriers for hard gelatin capsules. Suitable carriers for soft gelatin capsules are, for example, vegetable oils, waxes, fats, semi-solid and liquid polyols and the like. Depending on the nature of the active ingredient no carriers are, however, usually required in the case of soft gelatin capsules, other than the soft gelatin itself.

Capsule forms and methods of producing capsule forms, including hard and soft capsules such as gelatin capsules, are known in the art, for example as taught in U.S. Pat. Nos. 4,780,316; 6,759,395; 6,783,770; 6,967,026; and 7,025,911. The disclosure of each of these patents is incorporated herein by reference in their entireties.

The tablet and hard and soft capsule fomulations of the invention may also provide an early onset of therapeutic value through inclusion of an early onset layer of the opioid drug of interest. Early onset may be achieved, for example, through inclusion of an outer coating of the opioid drug designed to have rapid bioavailability. In this embodiment, at least 15% more of the drug will be released within the first 15, 30, 60, 75, or 90 minutes of administration, as compared to conventional forms of the same drug. In a preferred embodiment of the invention, substantially all of the early release of drug will occur in the gastric space, with only an insignificant amount of drug released in the oral cavity, in order to mask the taste of the drug.

### Methods of Preventing Drug Abuse and Treating Pain

The invention further contemplates methods of preventing drug abuse comprising administering to an individual an oral abuse-resistant drug dosage form comprising at least two populations of drug-resin complex particles, including a first population of drug-resin complex particles which is a sustained-release form comprising an analgesically effective amount of an opioid drug bound to a water-insoluble, pharmacologically inert matrix, the first population of drug-resin complex particles further comprising a water-permeable coating, and a second population of drug-resin complex particles comprising an aversive agent in an amount sufficient to deter abuse if tampered with, plus a polymeric coating which substantially prevents release of the aversive agent under normal use conditions.

The invention further contemplates methods of treating pain in an individual in need thereof using forms that prevent abuse by a drug abuser. In one embodiment, the invention contemplates methods of treating pain in an individual comprising administering to an individual suffering from a pain-related disease or disorder an oral abuse-resistant drug dosage form comprising at least two populations of drug-resin complex particles, including a first population of drug-resin complex particles which is a sustained-release form comprising an analgesically effective amount of an opioid drug bound to a water-insoluble, pharmacologically inert matrix, the first population of drug-resin complex particles further comprising a water-permeable coating, and a second population of drug-resin complex particles which comprises an aversive agent in an amount sufficient to deter abuse if tampered with, covered by a polymeric coating which substantially prevents release of the aversive agent under normal use conditions.

A non-limiting listing of pain-related diseases and/or disorders includes inflammatory pain, post-operative incision pain, complex regional pain syndrome, cancer pain, primary or metastatic bone cancer pain, fracture pain, chronic pain, osteoporotic fracture pain, pain resulting from burn, osteoporosis, gout joint pain, abdominal pain, pain associated with sickle cell crises, and other nociceptic pain, as well as hepatocellular carcinoma, breast cancer, liver cirrhosis, neurogenic pain, neuropathic pain, nociceptic pain, trigeminal neuralgia, post-herpetic neuralgia, phantom limb pain, fibromyalgia, menstrual pain, ovarialgia, reflex sympathetic dystrophy, neurogenic pain, osteoarthritis or rheumatoid arthritis pain, lower back pain, diabetic neuropathy, sciatica, or pain or visceral pain associated with: gastro-esophageal reflux, dyspepsia, irritable bowel syndrome, irritable colon, spastic colon, mucous colitis, inflammatory bowel disease, Crohn's disease, ileitis, ulcerative colitis, renal colic, dysmenorrhea, cystitis, menstrual period, labor, menopause, prostatitis, pancreatitis, renal colic, dysmenorrhea, cystitis, including interstitial cystitis (IC), surgery associated with the ileus, diverticulitis, peritonitis, pericarditis, hepatitis, appendicitis, colitis, cholecystitis, endometriosis, chronic and/or acute pancreatitis, myocardial infarction, kidney pain, pleural pain, prostatitis, pelvic pain, trauma to an organ, chronic nociceptive pain, chronic neuropathic pain, chronic inflammatory pain, fibromyalgia, breakthrough pain and persistent pain.

In another embodiment of the invention, the disease is cancer pain arising from malignancy or from cancer which may be selected from one or more of: adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, anemia, bladder cancer, bone cancer, brain cancer, breast cancer, carcinoid tumours, cervical cancer, chemotherapy, colon cancer, cytopenia, endometrial cancer, esophageal cancer, gastric cancer, head cancer, neck cancer, hepatobiliary cancer, kidney cancer, leukemia, liver cancer, lung cancer, lymphoma, Hodgkin's disease, lymphoma, non-Hodgkin's, nervous system tumours, oral cancer, ovarian cancer, pancreatic cancer, prostate cancer, rectal cancer, skin cancer, stomach cancer, testicular cancer, thyroid cancer, urethral cancer, bone cancer, sarcomas cancer of the connective tissue, cancer of bone tissue, cancer of blood-forming cells, cancer of bone marrow, multiple myeloma, leukaemia, primary or secondary bone cancer, tumours that metastasize to the bone, tumours infiltrating the nerve and hollow viscus, tumours near neural structures. Further preferably the cancer pain comprises visceral pain, preferably visceral pain which arises from pancreatic cancer and/or metastases in the abdomen. Further preferably the cancer pain comprises somatic pain, preferably somatic pain due to one or more of bone cancer, metastasis in the bone, postsurgical pain, sarcomas cancer of the connective tissue, cancer of bone tissue, cancer of blood-forming cells of the bone marrow, multiple myeloma, leukaemia, primary or secondary bone cancer.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described below.

These and other changes can be made to the invention in light of the above detailed description. In general, in the following claims, the terms used should not be construed to limit the invention to the specific embodiments disclosed in the specification and the claims.

The invention may be practiced in ways other than those particularly described in the foregoing description and examples. Numerous modifications and variations of the invention are possible in light of the above teachings and, therefore, are within the scope of the appended claims.

The entire disclosure of each document cited (including patents, patent applications, journal articles, abstracts, manuals, books, or other disclosures) in the Background of the Invention, Detailed Description, and Examples is herein incorporated by reference in their entireties.

### EXAMPLES

The following examples are put forth to supplement the preceding disclosure, so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Method of Preparing Oral Liquid Drug Form

An oral dosage form is produced by preparing two separate populations of drug-resin complex particles, the first population comprising an opioid drug and the second population comprising an opioid antagonist. Both populations utilize Amberlite IRP-69 resin particles, and are substantially similar in appearance.

The first population of drug-resin complex particles comprises the opioid oxycodone-HCl, while the second population of aversive agent-resin complex particles comprises the opioid antagonist naltrexone. In separate kettles, oxycodone-HCl and naltrexone are each mixed at a ratio of about 10:1, respectively, in purified water until dissolved. Amberlite IRP-69 is added to each kettle and mixed until the drug is equilibrated with bound drug on the resin.

Following mixing, both populations of particles are filtered through 20 um filters, followed by filtration through 8 um filters. The filtrate may be used to load a subsequent batch of resin. The loaded resinates are collected and returned to their respective kettles, where they are mixed to uniformity with polyethylene glycol.

Following mixing, both populations of particles are filtered through 20 μm filters, followed by filtration through 8 μm filters. The loaded resinates are then collected and oven dried until the Loss on Drying is between 3% and 7%. The dried material is then screened through a 100-mesh and 325-mesh screen. The material that passes through the 100-mesh screen but not the 325-mesh screen is then harvested and further processed.

The harvested first population of opioid drug-resin complex particles is then coated with a coat weight of about 30% by weight of the drug-resin complex particles of ethylcellulose in acetone, thereby providing a diffusion barrier coating. The harvested second population of aversive agent-containing resin complex particles comprising naltrexone is coated with a coat weight of about 50% by weight of the drug-resin complex particles of a poly(lactic/glycolic acid), a copolymer of lactic and glycolic acid. A coating at this coat weight substantially prevents release of the aversive agent under normal use conditions. This coating is applied at a 65:35 ratio of lactic acid to glycolic acid.

Following coating, the two populations of resin complex particles are combined in a liquid drug form. The liquid drug form is comprised of about 15% resin complex particles, wherein the ratio of the oxycodone-HCl-resin complex particles to naltrexone-resin complex particles is from about 4:1 to about 10:1, incorporated into an aqueous Avicel RC-591 suspending agent. The suspending agent further comprises about 30% water by weight/volume. The liquid drug form further comprises about 15% volume/volume of propylene glycol as a co-solvent. The liquid drug form further contains natural and/or artificial sweeteners, coloring agents, and flavoring agents as necessary to achieve a desired taste and appearance.

### Example 2

### Method of Treating Pain

The oral dosage form according to Example 1 is administered to a patient to provide pain relief. The oral dosage form comprises an orally effective amount of an opioid drug and an aversive agent that is in substantially non-releasable form consistent with the invention. The opioid agonist is formulated for release in the gastrointestinal tract.

When the untampered oral dosage form is administered orally and delivered to the gastrointestinal tract of a patient in need of pain therapy, the opioid drug is released from the dosage form during normal digestion, providing analgesia to the patient. Patients who take the oral dosage form without tampering (e.g. by mechanical agitation, heating, or dissolution in a solvent), will not have the aversive agent absorbed in sufficient amount during any time interval during the dosing of the oral drug dosage form such that the patient is denied the euphoric effect and/or is subjected to an aversive effect.

### Example 3

### Method of Preventing Abuse of an Opioid Drug

The oral dosage form according to Example 1 is used to prevent the abuse potential of an opioid drug contained therein. The oral dosage form comprises an opioid drug in combination with an aversive agent. The aversive agent is present in a form that is substantially non-releasable during digestion. However, when the oral dosage form is tampered with, e.g., by mechanical agitation (e.g., chewing, crushing, shearing, grinding), heat (e.g., temperatures of greater than 45° C., preferably between 45° C. to 50° C.), or dissolution of the dosage form in a solvent (with or without heating), the aversive agent is substantially released from the oral dosage form. Thus, when the dosage form is chewed, crushed, heated or dissolved in a solvent, and then administered orally, intranasally, parenterally or sublingually, the user is denied the euphoric effect and/or subjected to an aversive effect.

1. A sustained-release, abuse-resistant oral drug dosage form comprising at least two populations of drug-resin complex particles, wherein the first population of drug-resin complex particles is a sustained-release form comprising an analgesically effective amount of an opioid drug bound to a water-insoluble, pharmacologically inert matrix, said first population of drug-resin complex particles further comprising a water-permeable coating, and wherein the second population of drug-resin complex particles comprises an aversive agent in an amount sufficient to deter abuse if tampered with and a polymeric coating sufficient to substantially prevent release of the aversive agent under normal use conditions.

2. The drug dosage form of claim 1, wherein the opioid drug is selected from the group consisting of: alfentanil, allylprodine, alphaprodine, anileridine, benzylmorphine, bezitramide, buprenorphine, butorphanol, clonitazene, codeine, cyclazocine, desomorphine, dextromoramide, dezocine, diamorphone, diampromide, dihydrocodeine, dihydromorphine, dimenoxadol, dimepheptanol, dimethylthiambutene, dioxaphetyl butyrate, dipipanone, eptazocine, etorphine, dihydroetorphine, ethotheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metop on, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, prop eridine, propiram, propoxyphene, sufentanil, tramadol, tilidine, and mixtures of one or more of any of the foregoing.

3. The drug dosage form of claim 2, wherein the opioid drug is oxycodone, morphine (sulfate), oxycodone/acetaminophen, hydromorphone, tramadol hydrochloride, hydrocodone/acetaminophen, or codeine.

4. The drug dosage form of claim 2, wherein the opioid drug is in a salt form.

5. The drug dosage form of claim 1, wherein said aversive agent is an opioid antagonist selected from the group consisting of: naltrexone, nalmefene, cyclazacine, levallorphan, dextromethorphan ((+)-3-hydroxy-N-methylmorphinan), its metabolite dextrorphan ((+)-3-hydroxy-N-methylmorphinan), amantadine (1-amino adamantine), memantine (3,5 dimethylaminoadamantone), d-methadone (d-form of 6-dimethylamino-4, 4-diphenyl-3-heptanone hydrochloride), naloxone, etorphine, dihydroetorphine, and mixtures of one or more of any of the foregoing.

6. The drug dosage form of claim 1, wherein the opioid drug is prepared to include both a sustained release formulation and an immediate release formulation.

7. The drug dosage form of claim 1, wherein the drug-resin complex particles comprise a synthetic resin.

8. The drug dosage form of claim 1, wherein the drug-resin complex particles comprise a semi-synthetic resin.

9. The drug dosage form of claim 1, wherein the drug-resin complex particles are 30 to 500 microns in size.

10. The drug dosage form of claim 9, wherein the drug-resin complex particles are 40 to 150 microns in size.

11. The drug dosage form of claim 1, wherein the drug-resin complex particles are regularly shaped.

12. The drug dosage form of claim 1, wherein the first and second populations of drug-resin complex particles are substantially similar in one or more of size, color, appearance, and texture.

13. The drug dosage form of claim 1, wherein the first population of drug-resin complex particles comprises an enteric coating.

14. The drug dosage form of claim 1, wherein one or both of the populations of drug-resin complex particles further comprise a humectant.

15. The drug dosage form of claim 14, wherein the humectant is selected from the group consisting of: polyethylene glycol, propylene glycol, lactose, methylcellulose, hydroxypropylmethylcellulose, sorbitol, mannitol, polyvinylpyrrolidone, carboxypolymethylene, xanthan gum, propylene glycol, alginate, and mixtures of one or more of any of the foregoing.

16. The drug dosage form of claim 1, wherein the first population of drug-resin complex particles further comprises unbound opioid drug.

17. The drug dosage form of claim 1, wherein the first population of drug-resin complex particles comprises opioid drug at a concentration to provide about 1 mg to about 800 mg of opioid drug per 70 kg patient in a single dose.

18. The drug dosage form of claim 17, wherein the first population of drug-resin complex particles comprises opioid drug to provide about 10 mg to about 500 mg of opioid drug per 70 kg patient in a single dose.

19. The drug dosage form of claim 1, wherein the opioid drug is loaded onto the first population of drug-resin complex particles at a concentration of about 30% to about 60% by weight.

20. The drug dosage form of claim 1, wherein the water-permeable polymer coating is selected from the group consisting of: ethylcellulose, methylcellulose, hydroxypropylmethylcellulose (HPMC), hydroxyethlycellulose (HEC), acrylic acid ester, cellulose acetate phthalate, HEC phthalate, HPMC phthalate, and mixtures thereof.

21. The drug dosage form of claim 1, wherein the second population of particles further comprises one or more chelating agents.

22. The drug dosage form of claim 21, wherein the one or more chelating agents are administered between two coating layers covering the aversive agent.

23. The drug dosage form of claim 22, wherein the two coating layers covering the aversive agent comprise the same polymer coating.

24. The drug dosage form of claim 22, wherein the two coating layers covering the aversive agent comprise different polymer coatings.

25. The drug dosage form of claim 22, wherein the one or more chelating agents are selected from the group consisting of: EDTA, edetate calcium disodium, edetate trisodium, edetate disodium, edetate sodium, desferrioxamine B, deferoxamine, dithiocarb sodium, penicillamine, pentetate calcium, sodium salts of pentetic acid, succimer, trientine, nitrilotriacetic acid, trans-diaminocyclohexanetetraacetic acid (DCTA), diethylenetriaminepentaacetic acid, bis(aminoethyl)glycolether-N,N,N′,N′-tetraacetic acid, iminodiacetic acid, citric acid, tartaric acid, fumaric acid, salts thereof, and mixtures or combinations thereof.

26. The drug dosage form of claim 1, wherein the form is prepared as a liquid form.

27. The drug dosage form of claim 1, wherein the form is prepared as a tablet form, a capsule form, an orally disintegrating tablet (ODT) form, or a sprinkle form.

28. The drug dosage form of claim 1, wherein the drug dosage form is finally formulated.

29. A method of treating pain in an individual comprising administering the drug dosage form according to claim 1 to a subject in need thereof.

30. A method of preventing drug abuse, comprising administering the drug dosage form according to claim 1 to a subject in need thereof.
